(19) United States
(12) Patent Application Publication
(10) Pub. No.: US 2015/0231385 A1
(43) Pub. Date: Aug. 20, 2015

(54) LOW OPENING PRESSURE ANTI-SIPHON CHECK VALVE

(71) Applicant: ILLINOIS TOOL WORKS INC., Glenview, IL (US)

(72) Inventor: Jan Willem Marinus Mijers, Haarlem (NL)

(21) Appl. No.: 14/422,183

(22) PCT Filed: Aug. 20, 2013
(86) PCT No.: PCT/IB2013/001805

§ 371 Date: Feb. 18, 2015

(30) Foreign Application Priority Data
Aug. 20, 2012 (DE) 20 2012 007 884.4

(51) Int. Cl. A61M 39/24; A61M 39/26; A61M 5/36

(52) U.S. Cl. CPC A61M 39/24; A61M 5/36; A61M 39/26; A61M 2039/242; A61M 2039/2433; A61M 2039/2493; A61M 2039/261; A61M 2039/263; A61M 2039/266

(57) ABSTRACT

A one-way valve device, in particular a low-pressure check valve, for use in infusion equipment, with a housing having an inlet line and an outlet line, the inlet and outlet lines being connected to a pressure space in the housing, the inlet line being arranged on a first side of the housing, and the outlet line being arranged on a second side of the housing opposite the first side, the pressure space being constructed from two interconnected pressure chambers, and the one-way valve device being designed such that the through-flow is interrupted by a static underpressure prevailing on the inlet line and/or the outlet line or by an overpressure on the outlet line, wherein the two pressure chambers are arranged lying one above the other in the housing with respect to the flow direction between the first and the second side.

The invention relates to a one-way valve device, in particular a low-pressure check valve for use in infusion equipment, preferably for gravitational infusion and pump infusion, consisting of a housing with an inlet line and an outlet line, and with at least one seal lying in a pressure space.

Valve devices of the type in question are preferably used in infusion equipment in hospitals as a safeguard against running dry, wherein the valve devices are used to bring about an immediate stop to infusion when a reservoir filled with infusion liquid runs dry, so that no air gets into the infusion liquid and, toward the end of the delivery stream, into the venous system of a patient. A valve device for use in infusion equipment is known, for example, from the German published application DE 29 19 343 A1, which is equipped with a drip chamber and a float valve and which prevents entry of air into the infusion hose when the chamber runs dry. A further safeguard against running dry is known from the German published application DE 36 32 412 A1, in which a valve to protect against running dry is arranged upstream from a drip chamber in the direction of flow.

Moreover, DE 197 49 562 A1 discloses infusion equipment consisting of a highly suspended container for the infusion liquid and of a drip that can be connected to a throttle container by means of a mandrel, an adjustable roller clamp on a flexible inlet line beneath the drip, and an injection needle located at the line end. To prevent entry of air into the infusion liquid, in particular toward the end of the delivery of infusion liquid, a safeguard against running dry is provided upstream from the drip chamber in the direction of flow, or on the admission opening of the drip chamber, and the built-in valve thereof can sop the infusion liquid in a manner dependent on a prevailing static pressure of the through-flow. However, it has been found in practice that a safeguard against running dry as a function of the static pressure of the infusion liquid is not adequate to prevent entry of air, particularly if there is a possibility of the connections being mixed up through carelessness and, therefore, of the function of the safeguard valve no longer being guaranteed.

This is particularly dangerous if air gets into the system and advances into the patient's veins, where it can lead to an embolism. It is therefore important that the infusion liquid does not entrain any air which leads to the aforementioned disadvantage. A particular disadvantage is that, if there is a static overpressure at the outlet side, the previously known valve cannot prevent entry of air into the system.

Finally, DE 102 19 994 A1 discloses a one-way valve device which tackles the disadvantages of the aforementioned prior art and proposes a valve device in which the entry of air into the system of the infusion equipment can be prevented with greater reliability. For this purpose, provision is made that the through-flow is interrupted when a static underpressure arises on the inlet and/or outlet line or when there is an overpressure on the outlet line. For the purpose of sealing the inlet line and the outlet line, a seal is provided which, inside a pressure space, bears alternately on one side on a first sealing ring lip and on the opposite side on a second sealing ring lip, in each case with tensioning. The seals obtained in this way provide sealing of the sealing ring lips, which are operatively connected to the inlet line and outlet line and, in the event of an underpressure, permit sealing with respect to the sealing ring lips. On account of the alternate sealing of the sealing ring lips by at least one seal, it is ensured that, in the event of an overpressure on the outlet line, a lifting of the seal takes place only on the sealing ring lip assigned to the outlet line, whereas by contrast the seal on the second sealing ring lip is pressed on account of the prevailing overpressure, thereby closing the inlet line. As an alternative to the use of a single pressure space with a seal located therein, that is to say a single membrane, it is also possible that two pressure chambers communicating via a connecting channel are formed, and a two-part seal is used.

A disadvantage of the proposed valve device is that the overall structure necessitates an unreasonable size and, particularly when the valve device is combined with an injection pump, this can reach such a considerable extent that there is a risk of the whole device accidentally being broken off.

As a result of the large dimensions, the costs of the valve device are also quite high. The costs of the special rubber membrane are also quite high, and, because of their complexity, the membrane or the valve device as a whole cannot easily be produced in an automated manufacturing method.

There is also the disadvantage that the opening pressure of the valve device is quite high and, for reasons relating to the appliance, cannot be reduced. The opening pressure is approximately the pressure of a 50 cm water column. Accordingly, the use of the valve device is suitable only in combination with infusion pumps, but not with those that operate by gravitational pressure.

The object of the invention is therefore to make available an improved one-way valve device that eliminates the aforementioned disadvantages of the prior art in DE 102 19 994 A1.

According to the invention, and in contrast to the valve device proposed in DE 102 19 994 A1, the object is achieved by the fact that, instead of two pressure chambers using the same membrane for the respective sealing and being arranged in the same plane, two pressure chambers are arranged one after the other in the flow direction of the medium that is to be administered. This means that the two pressure chambers lie with their bases over each other. This affords the advantage that the valve device can have much smaller dimensions, particularly as regards its width as seen in relation to the flow direction. Therefore, the two pressure chambers do not lie in the same plane but in two different planes that are preferably parallel to each other. It is desirable that the two pressure chambers at least partially overlap each other in a plan view. Plan view signifies the flow direction, or the direction from one base side of the cylinder of the housing to the other side, or from the first side of the housing to its second side.

For example, the valve device can thus have a cylindrical shape, wherein the first pressure chamber is arranged in the upper area of the cylinder (if the cylinder is standing on an end face) and the second pressure chamber in the lower area.

By arranging the two pressure chambers in tandem, the opening pressure can also be much better adjusted and can reach lower values, such that, for example, infusion equipment with gravitational pressure can be used in combination with the proposed valve device.

In addition, there is also the advantage that the proposed valve device can be more easily used with needle-free valves as are offered presently on the market.

Although more components are needed by comparison with the prior art, these components can be produced and assembled more easily, such that the production costs as a whole can be reduced. Moreover, the time needed for production can be reduced, since the components generally are of less complexity.

In the valve device according to the invention, it is necessary that at least two seals or membranes are used. According to the invention, these are likewise arranged in tandem with respect to the flow direction of the medium that is to be administered. Therefore, the two seals or membranes do not lie in the same plane but instead in two different planes that are preferably parallel to each other.

It is desirable that the two seals at least partially overlap each other in the plan view.

As regards its function, however, the valve device according to the invention corresponds in other respects to the device known from DE 102 19 994 A1.

It is further proposed that the housing of the valve device according to the invention has a main part and a housing attachment. These two parts are intended to be joined and closed so as to be leaktight. This can be done, for example, by means of ultrasonic welding when connecting the two parts to each other. The housing attachment preferably has an inlet line into the valve device, and the main part has an outlet line from the valve device for the medium that is to be administered. A first sealing ring lip is operatively connected to the inlet line, and a second sealing ring lip is operatively connected to the outlet line.

The assembled parts of the housing preferably form a hollow space in which the seals or membranes are arranged. Moreover, an inner valve part can be arranged in the hollow space, preferably between the two seals or membranes, and can serve for securing the seals or membranes and/or for providing a seal together with the seals or membranes and/or for conveying the medium that is to be administered through the valve device.

The first and second pressure chambers are also arranged in the aforementioned recess between the main part and the housing attachment of the housing. The walls of the first and second pressure chambers are therefore defined by the inner part, and the first and/or second seal, and they can preferably also be defined by the main part or the housing attachment.

The first seal is preferably part of a first valve disk, and the second seal is preferably part of a second valve disk. The valve disks and/or the seals or membranes can have through-holes.

The first valve disk is preferably clamped between the inner housing part and the housing attachment in such a way that the seal or membrane bears with pretensioning against the housing attachment. Alternatively, the seal or membrane can also bear with pretensioning against the valve disk.

The second valve disk is preferably clamped between the inner housing part and the main part in such a way that the seal or membrane bears with pretensioning against the inner face of the second valve disk and/or against the main part.

Thus, the same technical effects as in the prior art in DE 102 19 994 A1 can be achieved, namely that the through-flow is interrupted when a static underpressure arises on the inlet line and/or outlet line or when there is an overpressure on the outlet line. A static underpressure on the inlet line can arise, for example, when a reservoir containing the infusion liquid, or the medium to be administered, has run dry, when an infusion pump is operating incorrectly or when air has entered the system. An overpressure on the outlet line can arise, for example, upon temporary closure of the arm veins of the patient, when a medicament pump is additionally attached on the output side. An overpressure would lead to the medicament being pumped into the gravitational infusion system.

However, the valve device according to the invention ensures that, in the aforementioned cases, further transport of the infusion liquid is reliably stopped and, consequently, the entry of air-enriched infusion liquid into the veins is prevented. In addition, the one-way valve ensures that, when an overpressure prevails on account of an infusion pump or medicament pump, the valve closes on the output side and, consequently, the medicament does not get into the infusion system, as a result of which a transient overdose is prevented.

For sealing purposes, sealing ring lips or sealing lips are preferably used in addition to the seals or membranes. They can be arranged on the parts of the housing, that is to say, for example, on the housing attachment and/or on the main part. The seals or membranes preferably bear with pretensioning against the sealing ring lips.

Alternatively, it is conceivable that the sealing ring lips are arranged on the seals or membranes. They can be arranged such that the seals or membranes press with the sealing ring lips against the parts of the housing, that is to say the first seal or membrane with its sealing ring lip against the housing attachment and/or the second seal or membrane with its sealing ring lip against the main part.

This embodiment has the advantage that the seals or membranes are therefore slightly stiffer. An even lower opening pressure of the valve device can thereby be achieved.

The invention is explained in more detail below with reference to the figures.

In the figures:

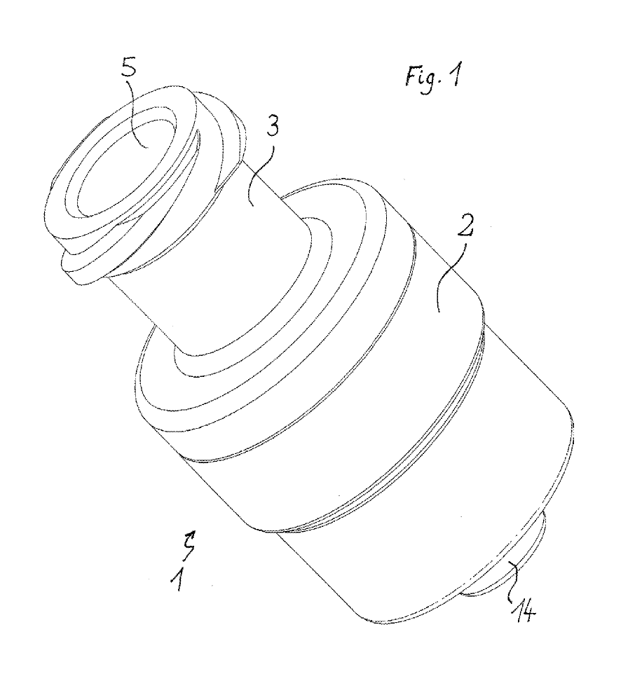

FIG. 1 shows a perspective view of a one-way valve device according to the invention;

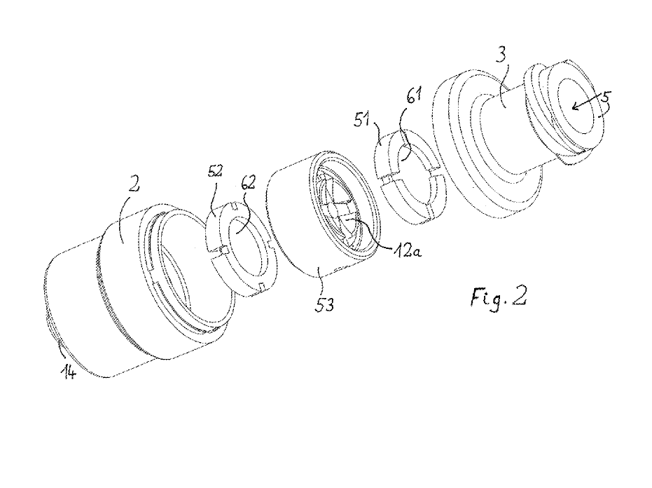

FIG. 2 shows an exploded view of the one-way valve device from FIG. 1;

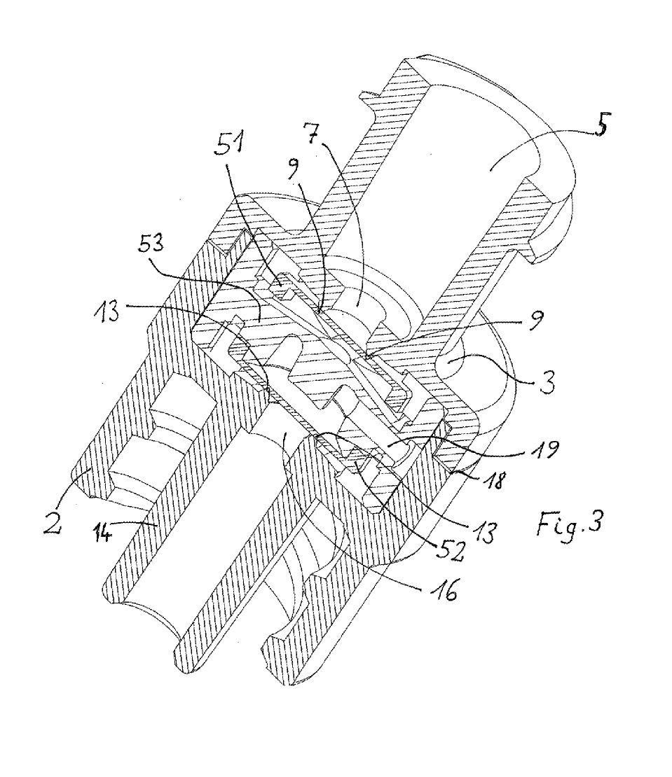

FIG. 3 shows a cross-sectional, perspective view of the one-way valve device according to a first embodiment;

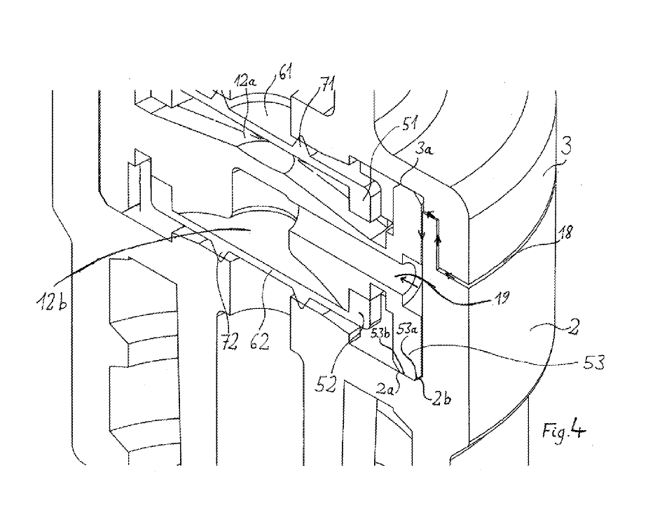

FIG. 4 shows a cross-sectional, perspective detail of the housing;

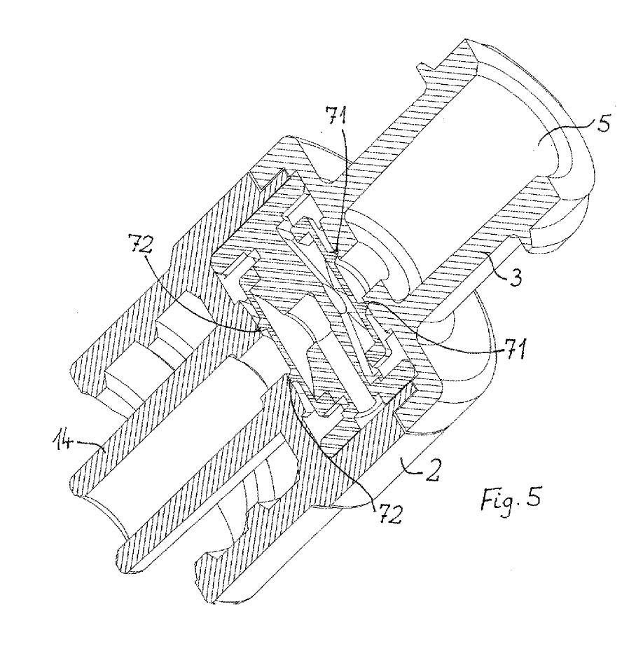

FIG. 5 shows a cross-sectional, perspective view of a valve device according to a second embodiment;

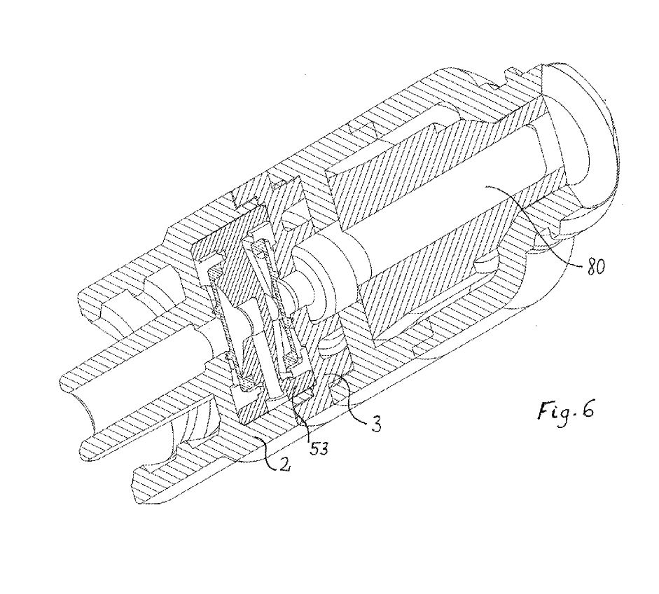

FIG. 6 shows a cross-sectional, perspective view of a valve device according to the invention, which is connected to a needle-free appliance;

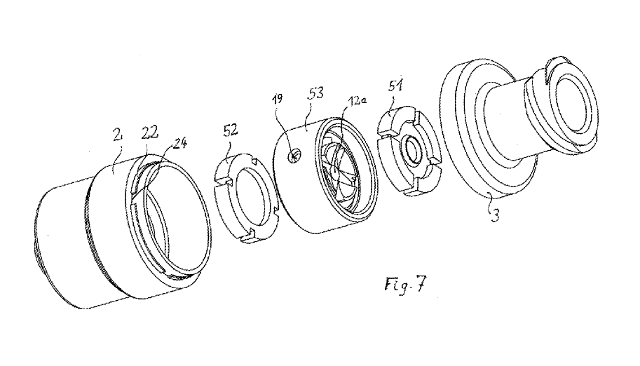

FIG. 7 shows an exploded view of the overall appliance from FIG. 6;

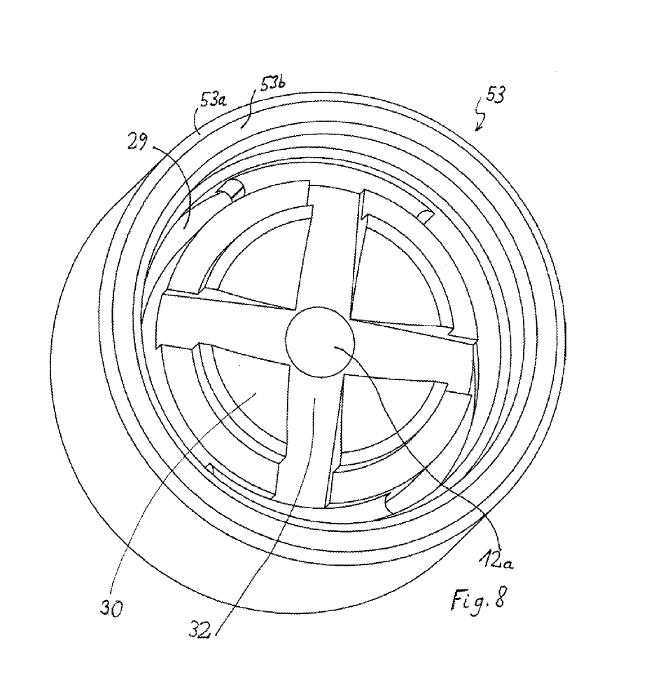

FIG. 8 shows a detailed view of the inner housing part 53 of the one-way valve device according to the invention from FIG. 1;

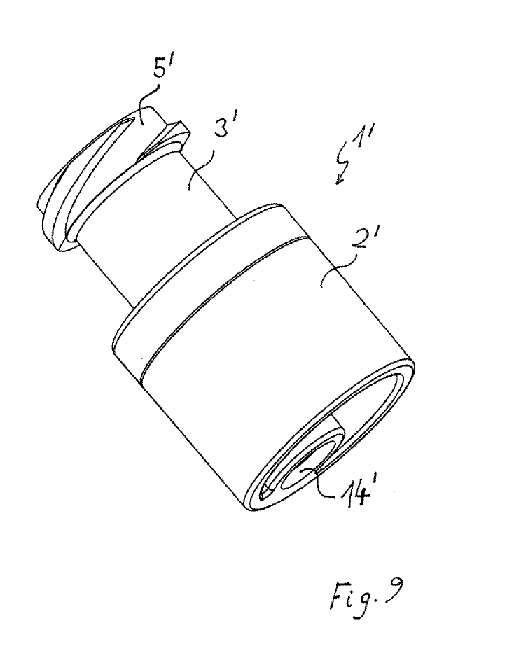

FIG. 9 shows a further embodiment of a one-way valve device according to the invention;

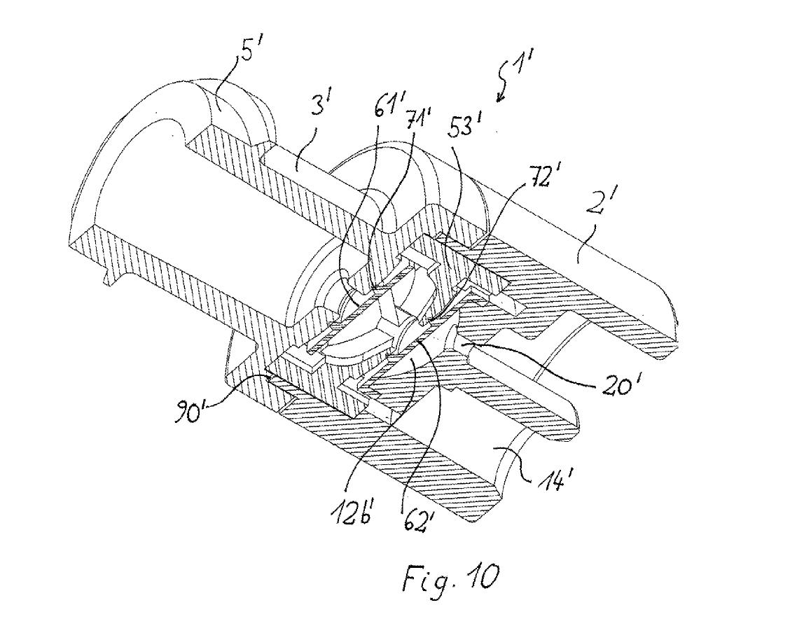

FIG. 10 shows a cross-sectional view of the one-way valve device according to the invention from FIG. 9;

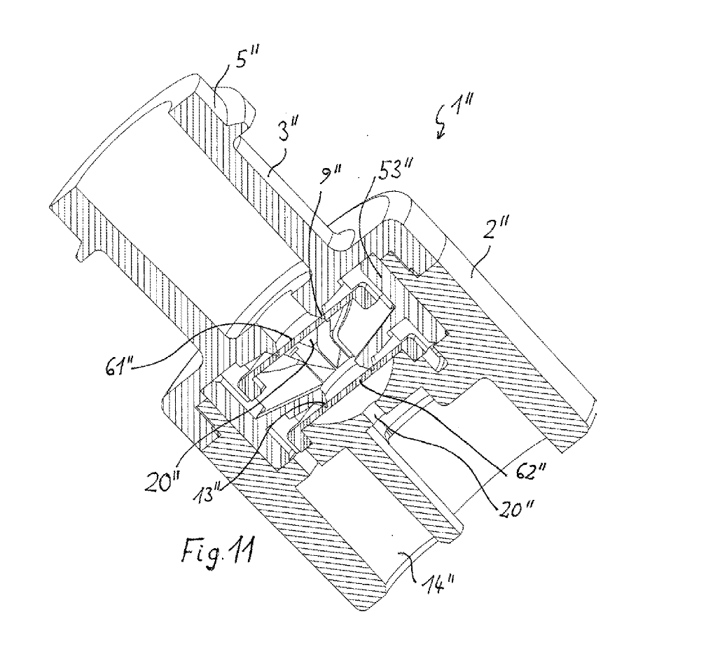

FIG. 11 shows a modification of the one-way valve device according to the invention from FIGS. 9 and 10;

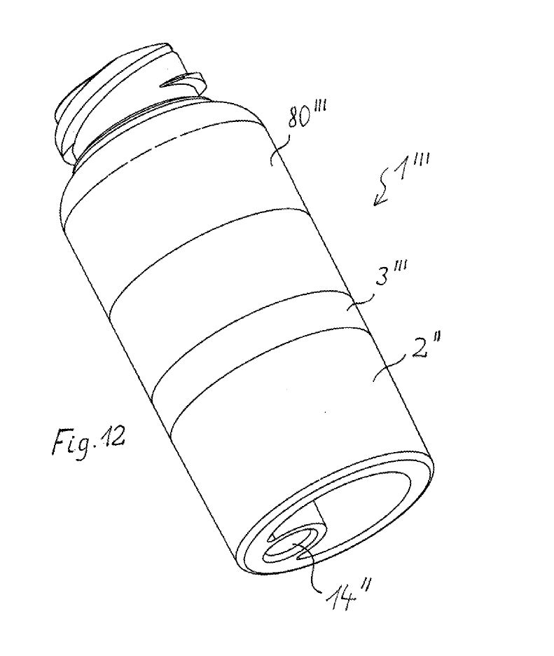

FIG. 12 shows a further modification of a one-way valve device according to the invention with a needle-free appliance, and

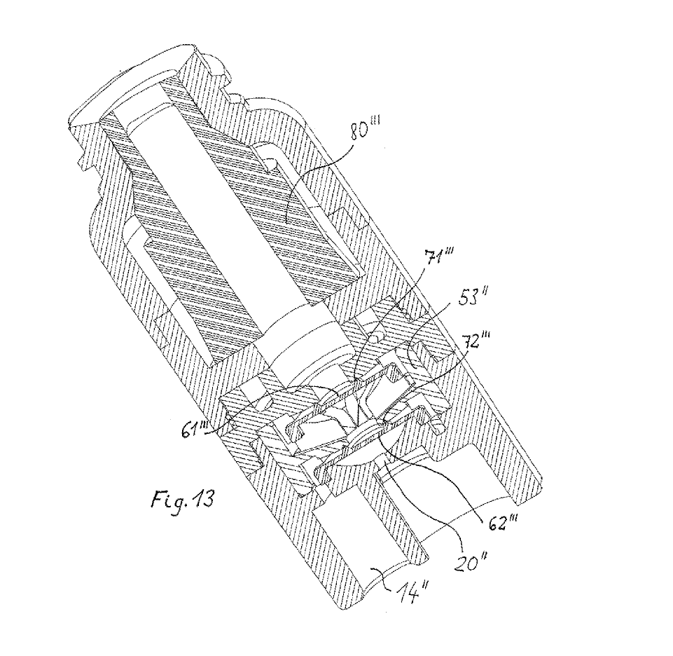

FIG. 13 shows a cross-sectional view of the one-way valve device according to the invention from FIG. 12.

The one-way valve device 1 according to the invention shown in FIG. 1 has an approximately cylindrical shape overall. The approximately cylindrical housing of the valve device 1 comprises the main part 2 and the housing attachment 3 arranged on the latter. The housing attachment 3 comprises the inlet line 5, while the main part 2 has the outlet line 14. The inlet line 5 and outlet line 14 are arranged on the opposite end faces of the cylindrical housing 2, 3. Preferably, as can be seen from FIG. 1, the inlet line and outlet line protrude perpendicularly from the bases of the cylindrical housing. However, it is also conceivable that they protrude at an angle or parallel to the respective end face.

FIG. 2 shows an exploded view of the valve device 1 according to the invention. The individual parts are each shown in their respective order inside the valve device 1 with respect to the flow direction of the medium that is to be administered, which basically leads from the inlet line 5 in the direction of the outlet line 14.

Two valve disks 51, 52, with an inner housing part 53 lying between them, are arranged inside the housing 2, 3, that is to say substantially within the receiving area inside the main part 2. The first valve disk 51, seen in the flow direction, has at least one seal or membrane 61. The inner housing part 53 is arranged downstream from this in the flow direction. Downstream from the inner housing part 53 is the second sealing disk 52, which has at least one seal or membrane 62.

FIG. 3 shows a perspective cross-sectional view of a valve device according to a first embodiment, in the form when assembled. As can be seen, the inner housing part is designed in relation to the main part 2 and to the housing attachment 3 such that the seal 61 of the first valve disk 51 is pressed in between the inner housing part 53 and the housing attachment 3. The first valve disk 51 and/or the first seal or membrane 61 is designed such that it bears with pretensioning on a sealing ring lip 9, which is located on the housing attachment 3. Sealing can thereby be ensured when an underpressure is present inside the inlet line 5 within the housing attachment 3. Sealing can thereby also be provided in the normal state, i.e. when the valve device is not in operation and the pressure is equalized throughout.

The sealing ring lip 9 preferably surrounds an aperture 7 inside the housing attachment 3, which is part of the inlet line 5. The sealing ring lips are preferably designed in principle in an annular shape. This annular shape preferably surrounds the aperture 7 of the inlet line and the opening 16 of the outlet line.

In the same way, sealing is provided by the second valve disk 52 and/or by the second seal or membrane 62 and a second sealing ring lip 13, which is arranged on the main part 2 of the housing. The sealing ring lip 13 surrounds an opening 16, which is part of the outlet line 14. The valve disk 52 and the seal 62 are pressed again with pretensioning onto the sealing ring lip 13. The valve disk 52 thus sits, in a manner corresponding to the first valve disk 51, with an interference fit between the inner housing part 53 and the main part 2 of the housing. The second valve disk 52 thus provides sealing when the pressure in all areas of the valve device is equalized (i.e. when not in operation, for example) and when there is an underpressure in the outlet line 14, which is located inside the main part 2 of the housing.

In this embodiment of the invention, the relevant reference pressure is made available via a continuous slit 18 between the housing parts 2 and 3 and a channel 19 in the inner valve part 53. In the present invention, this pressure space 12 is divided into a pressure chamber 12a below the membrane 61 and a pressure chamber 12b below the membrane 62. Both pressure chambers 12a, 12b form the pressure space and are connected to each other and to the outside air as described. The first pressure chamber 12a, which is located inside the inner housing part 53, downstream from the first valve disk 51 in relation to the flow direction, is additionally designed such that, when an overpressure is present therein, the seal or membrane 61 is pressed onto the sealing ring lip 9 and thus closes. Thus, for example, an overpressure on the outlet line 14, as can occur for example when the arm veins of the patient are temporarily closed, cannot lead to the supplied medium flowing back again through the valve device counter to the flow direction.

FIG. 4 shows a perspective detail of the housing 2, 3 of the valve device according to the invention. Arrows shown in the figure indicate a channel extending from a housing opening 18 to the pressure chamber 12b. The latter is delimited by the second valve disk 52 on one side. The pressure chamber 12b is thus arranged lying opposite that side of the valve disk 52 that is oriented in the direction of the outlet line 14. The pressure compensation inside the pressure chamber 12b with atmospheric pressure has the effect that the pressure chamber 12b, and therefore the corresponding side of the valve disk 52, i.e. of the seal 62, is exposed as standard to atmospheric pressure. This ensures, on the one hand, that the seal 62 can be lifted from the sealing ring lip 13 by a medium flowing through the valve device (which leads to displacement of the air from the pressure chamber 12b), as a result of which a through-flow is permitted, and, on the other hand, that the seal 62 is able to press against the sealing ring lip 13 when there is an underpressure in the outlet line 14 (which leads to air being sucked into the pressure chamber 12b).

According to the invention, the housing opening 18 is obtained in a particularly simple way if the ultrasonic welding between the main part 2 and the housing attachment 3 is not carried out over the entire circumference of the housing and, instead, sectors are omitted. In this area, a space then remains between the two housing parts 2 and 3, which space forms this housing opening 18. It is particularly preferable if the inner housing part 53 is pressed with an interference fit between a circular plane area 2a of the main part 2 and a similarly plane area 3a of the housing attachment 3. In this way, it is at the same time ensured that the air-carrying connection between the housing opening 18 and the pressure chamber 12 is sealed off in relation to the fluid-carrying parts of the valve. For this purpose, it is particularly preferable for the inner housing part 53 to be made of a thermoplastic elastomer with a Shore hardness of 70 to 80 A.

Moreover, a perfect centering of the inner housing part 53 in the main part 2 of the housing, and therefore also in the housing attachment 3, can be achieved if the inner housing part 53 has, on its outer edges between end face and circumferential face, a bevel 53a, which corresponds to a matching bevel 2b at the transition between the perpendicular housing wall of the main part 2 and the circular plane surface 2a of the main part 2. In this way, when the housing parts 2 and 3 are joined together, the inner housing part 53, inserted beforehand between these parts, is optimally centered automatically and, therefore, a correct position of the valve lips on their mating surfaces is also achieved.

FIG. 5 shows a perspective cross-sectional view of the valve device according to the invention in a second embodiment. This is similar to the embodiment from FIG. 3, except that the sealing ring lips 71, 72 are not arranged on the housing parts but instead on the seals or membranes 61, 62. The sealing ring lips 71, 72 are arranged such that they are each pressed onto the corresponding housing parts 2, 3 as a result of the pretensioning inside the seals or valve disks. Therefore, whereas the sealing ring lips 9, 13 in the first embodiment are arranged on opposite sides in the recess defined by the housing 2, 3 and face each other, the sealing ring lips 71, 72 in the second embodiment are arranged on the seals 61, 62 and point away from each other.

FIGS. 6 and 7, finally, show an illustrative use of the valve device 1 according to the invention. In this illustrative use, the valve device according to the invention is used together with a standard needle-free appliance. The needle-free appliance 80 is secured on the housing attachment 3. The housing attachment 3 can therefore be adapted to the appliance that is to be secured thereon. The valve device according to the invention can therefore be used variably since, in different applications, the valve device can have basically the same construction and it is only the housing attachment 3 that has to be adapted.

The exploded view in FIG. 7 shows very nicely how the housing opening 18 is obtained, that is to say how the pressure chamber 12 is obtained via a channel 20 and the space between the housing parts 2 and 3. As can be seen from the view of the housing main part 2 in this exploded view, strips of material 22 for the ultrasonic welding of the two housing parts 2, 3 are provided in an outer bevel of the housing main part 2, onto which the housing cover 3 is fitted. These strips do not extend about the entire circumference and instead each cover circle sectors of considerably less than 90°. Between them, gaps 24 are provided which ensure the air connection of the pressure chamber 12 via the channel 20 and the through-opening 18 formed between housing main part 2 and housing cover 3.

A further possible use lies in injection pumps and infusion applications. The valve device according to the invention is in fact designed such that, even with a very low opening pressure (generally between 1.5 and 5 Psi or between 104 and 345 millibar) and a very low infusion volume with an infusion speed of 0.2 to 1 mm³ per hour, it permits a constant through-flow, prevents backward flow of the medium and can prevent runoff during exchange of the supply containers or injection devices (pumps, etc.) and can prevent air from being fed into the veins of the patient.

A further area of use of the valve device according to the invention lies in infusion systems that operate with gravitational pressure. The one-way valve device according to the invention can in fact be designed such that it already opens at a pressure of a water column of 15 cm.

Finally, the use of peristaltic pump infusion is also possible in the valve device according to the invention. In this case, however, it is important that the valve device is arranged directly downstream from the pump in the flow direction and not at the distal end (for example of a hose arranged between the pump and the valve device). In the latter case, the required liquid column downstream from the valve device, which is needed for the correct functioning of the anti-siphon valve, would be absent. It would then be possible for air to enter the infusion equipment.

FIG. 8 shows a detail of the inner housing part 53 of the above-described one-way valve device 1 according to the invention, seen from the inflow side, i.e. from the direction of the inlet line 5, such that it is mainly the pressure chamber 12a that can be seen here. In its edge area, this pressure chamber 12a is connected by arc-shaped through-openings 29 to the other pressure chamber 12b and is connected via the latter and the connecting channel 19 to the outside air pressure, as has been explained in the description of FIG. 4.

In the direction of flow, this pressure chamber 12a is delimited by an abutment face for the membrane 61. This is formed by four circle sectors 30, which each rise outward in a concave curvature and thus prevent overloading of the membrane, since they support this membrane 61 in the curved state.

To be able nonetheless to subject the membrane 61 to outside air pressure, the sectors 30 are each separated by rectangular channels 32, which become deeper toward the outside.

FIG. 8 also shows very clearly the bevel 53a on the outer edge of the inner housing part 53, which bevel 53a then merges into the plane face 53b that is needed for the interference fit of the inner housing part 53 between housing main part 2 and housing attachment 3.

FIG. 9 describes a further embodiment of the one-way valve device according to the invention. This design has the disadvantage of not being rotationally symmetrical, but it nonetheless has the advantage that it does not require admission of the external air pressure from the side, such that it can also be enclosed, for example, in a hose or wrapped in a plastic film. On its input side, this embodiment of the present invention also has an approximately cylindrical shape, in the same way as in the embodiment shown in FIG. 1. The approximately cylindrical housing of the valve device 1′ also has a main part 2′ and, arranged on the latter, a housing attachment 3′ which likewise has an inlet line 5′ with an external thread. In this embodiment, however, the outlet line 14′ is arranged eccentrically.

The structure of this further embodiment of the one-way valve 1′ according to the invention is explained in more detail below with reference to FIG. 10. The latter shows a cross-sectional view along the rotation axis of the cylindrical housing 2′, 3′.

As can be seen in FIG. 10, an inner valve part 53′ is also received here between the housing main part 2 and the housing attachment 3′ and held with an interference fit. Here, however, the inner housing part 53′ can be made much simpler, since it does not have to produce a lateral connection with the external atmosphere. Rather, the fluid outlet line 14′ is arranged eccentrically here, and the remaining space serves to produce a pressure compensation channel 20 arranged parallel to the outlet line 14′ and leading from the outlet end face of the housing main part 2 into the housing, which pressure compensation channel 20 leads directly into the pressure chamber 12b′ on the outlet end. In contrast to the embodiment described first, both membranes 61′ and 62′ here work in the same direction and not against each other. Here, an embodiment is once again shown in which the corresponding sealing lips 71′, 72′ are mounted on the membranes and work on smooth valve seats on the housing attachment 3′ and on the inner housing part 53′.

Of course, this embodiment too can be modified in such a way that the membranes 61′, 62′ are each smooth, and corresponding lips 9′, 13′ are formed on the valve seats, as is shown in FIG. 11 in a cross-sectional view of a correspondingly modified embodiment of the one-way valve device 1′.

Moreover, FIG. 11 also shows a view in which the support for the membrane 61″ is provided by quite a large number of narrower support elements 20″ shaped as sectors of a circle.

In the embodiment according to FIGS. 10 and 11, no lateral admission of air is needed. Therefore, the housing main part 2′, 2″ and the housing attachment 3′, 3″ can be connected by an ultrasonic welding seam 90′, 90″ running completely about the circumference, and this of course makes production easier.

FIG. 12 shows a further embodiment of the configuration according to the invention shown in FIGS. 9 to 11, but this time for a needle-free appliance.

Here too, as in the embodiment according to FIGS. 6 and 7, a needle-free appliance 80′″ is mounted on a specially modified housing attachment 3′″.

The configuration of the other parts of the device according to the invention corresponds to the eccentric arrangement of FIGS. 9 to 11, that is to say the main part 2′ of the housing with the outlet opening 14′ corresponds to the configuration in FIGS. 9 to 11.

FIG. 13 shows a cross-sectional view of the embodiment from FIG. 12, sectioned along the rotation axis of the cylindrical outer shape.

The cross-sectional view in FIG. 13 clearly shows the correspondence to the cross-sectional view in FIG. 11 in the area of the main part 2″of the housing and of the inner housing part 53″.

In this embodiment, however, the configuration has once again been chosen in which the sealing lips 71′″ and 72′″ are arranged on the respective membranes 61″ and 62′″, respectively, and not on the respective valve seat, which accordingly has a plane shape in this case.

### CITATIONS INCLUDED IN THE DESCRIPTION

This list of documents cited by the applicant has been generated automatically and is included solely to better inform the reader. The list is not part of the German patent or utility model application. The German Patent and Trademark Office accepts no liability for any errors or omissions.

### Cited Patent Literature

DE 2919343 A1 [0002]
DE 363241 A1 [0002]
DE 19749562 A1 [0003]
DE 10219994 A1 [0005, 0009, 0010, 0017, 0024]

1. A one-way valve device, in particular a low-pressure check valve, for use in infusion equipment, with a housing having an inlet line and an outlet line, the inlet and outlet lines being connected to a pressure space in the housing, the inlet line being arranged on a first side of the housing, and the outlet line being arranged on a second side of the housing opposite the first side, the pressure space being constructed from two interconnected pressure chambers, and the one-way valve device being designed such that the through-flow is interrupted by a static underpressure prevailing on the inlet line and/or the outlet line or by an overpressure on the outlet line, wherein the two pressure chambers are arranged lying one above the other in the housing with respect to the flow direction between the first and the second sides.

2. The one-way valve device as claimed in claim 1, wherein the two pressure chambers are arranged lying one above the other in the housing in such a way that their bases at least partially overlap in a plan view.

3. The one-way valve device as claimed in claim 1, wherein at least two seals or membranes separating the inlet line from the outlet line are provided in the pressure space, and the two seals or membranes are arranged lying one above the other in the housing with respect to the flow direction between the first and second sides.

4. A one-way valve device, in particular a low-pressure check valve, for use in infusion equipment, with a housing having an inlet line and an outlet line, the inlet and outlet lines being connected to a pressure space in the housing, the inlet line being arranged on a first side of the housing, and the outlet line being arranged on a second side of the housing opposite the first side, at least two seals or membranes separating the inlet line from the outlet line being provided in the pressure space, and the one-way check valve being designed such that the through-flow is interrupted by a static underpressure prevailing on the inlet line and/or the outlet line or by an overpressure on the outlet line, wherein the two seals or membranes are arranged lying one above the other in the housing with respect to the flow direction between the first and second sides.

5. The one-way valve device as claimed in claim 1, wherein the two pressure chambers are arranged lying one above the other in the housing in such a way that their bases at least partially overlap in a plan view.

6. The one-way valve device as claimed in claim 1, wherein the pressure space is designed as two interconnected pressure chambers, and the two pressure chambers are arranged lying one above the other in the housing with respect to the flow direction between the first and second sides.

7. The one-way valve device as claimed in claim 1, wherein the housing has a main part and a housing attachment.

8. The one-way valve device as claimed in claim 1, wherein the one-way valve device has a first valve disk and second valve disk, which each comprise at least one of the seals or membranes, and has an inner valve part, said valve disks and inner valve part being received in a recess between the main part and the housing attachment, and the inner valve part being arranged between the valve disks.

9. The one-way valve device as claimed in claim 1, wherein the first valve disk is clamped between the inner housing part and the housing attachment in such a way that the seal or membrane bears with pretensioning against the housing attachment, and/or the second valve disk is clamped between the inner housing part and the main part in such a way that the seal or membrane bears with pretensioning against the inner face of the second valve disk and/or against the main part.

10. The one-way valve device as claimed in claim 1, wherein at least one of the two seals or membranes bears with pretensioning against a corresponding sealing ring lip, which is arranged on the housing attachment and/or the main part and/or on the first and/or second valve disk.

11. The one-way valve device as claimed in claim 1, wherein at least one of the two seals or membranes has a sealing ring lip, which bears with pretensioning on the corresponding part of the one-way valve device.

12. The one-way valve device as claimed in claim 1 wherein at least one of the two valve disks has a through-hole.

13. The one-way valve device as claimed in claim 1, wherein the housing has a cylindrical shape, and the first and second sides form the end faces of the cylinder.

14. The one-way valve device as claimed in claim 1, wherein the inlet line is arranged perpendicularly with respect to the first side of the housing and/or the outlet line is arranged perpendicularly with respect to the second side of the housing.

15. The one-way valve device as claimed in claim 1, wherein the pressure space is designed as two pressure chambers connected to each other via a connecting channel, and the connecting channel opens out once above and once below the seals or membranes.

16. The one-way valve device as claimed in claim 1, wherein the inlet line opens into a pre-chamber in which the first sealing ring lip is designed with a closed hollow, on which the seal or membrane delimiting the pressure space bears.

17. The one-way valve device as claimed in claim 1, wherein the outlet line opens out inside the second sealing ring lip in the pressure chamber of the housing on which the seal or membrane delimiting the pressure space bears.

18. The one-way valve device as claimed in claim 1, wherein a second pre-chamber, delimited by the seal or membrane, is provided opposite the second sealing ring lip, which pre-chamber is exposed to atmospheric pressure through a housing opening.

19. The one-way valve device as claimed in claim 1, wherein the seal or membrane has, in the area of the first sealing ring lip, an opening which is designed lying centrally inside the first sealing ring lip, in such a way that the pressure space is sealed off in relation to the pre-chamber.

20. The one-way valve device as claimed in claim 1, wherein the surfaces enclosed by the first and second sealing ring lips are designed smaller than the respectively remaining surfaces of the bearing seal or membrane.

21. The one-way valve device as claimed in claim 3, wherein the opening or closing pressure is adjustable by the ratio of the enclosed surfaces inside the sealing ring lips to the remaining surfaces of the bearing seal or membrane.

22. Infusion equipment, having a one-way valve device as claimed in claim 1 and a pump, wherein the one-way valve device is arranged directly downstream from the pump in the direction of flow of the medium that is to be pumped.
